SEQUENCE LISTING (2 sequences)

SEQ ID NO: 1 - DNA, 22 bp, Artificial Sequence
  Primer
agagggacatccaggactttgt

SEQ ID NO: 2 - DNA, 20 bp, Artificial Sequence
  Primer
caggcgcttgttgggtgtac

(19) United States
(12) Patent Application Publication
(10) Pub. No.: US 2018/0200279 A1
(43) Pub. Date: Jul. 19, 2018

(54) INHIBITION OF HSV-1-ASSOCIATED CORNEAL NEOVASCULARIZATION USING INHIBITORS OF CYCLIN-DEPENDENT KINASE 9

(71) Applicant: XAVIER UNIVERSITY OF LOUISIANA, New Orleans, LA (US)

(72) Inventor: Harris E MCFERRIN, New Orleans, LA (US)

(21) Appl. No.: 15/329,964

(22) PCT Filed: Jul. 30, 2015
(86) PCT No.: PCT/US2015/042802

§ 371 Date: Jan. 27, 2017

Related U.S. Application Data
(60) Provisional application No. 62/031,443, filed on Jul. 31, 2014.

(51) Int. Cl. A61K 31/7056; A61K 31/453; A61P 27/02; A61P 35/00

(52) U.S. Cl. CPC A61K 31/7056; A61P 35/00; A61P 27/02; A61K 31/453

(57) ABSTRACT

Provided are methods of preventing and/or treating herpes simplex virus-1 (HSV-1)-associated ocular neovascularization comprising administering a cyclin-dependent kinase (CDK) inhibitor. Preferably a CDK9 inhibitor, such as flavopiridol or dichlorobenzimidazole-1-β-D-ribofuranoside (DRB), is administered to prevent and/or treat HSV-1-associated ocular neovascularization. Additionally, methods of inhibiting angiogenesis and HSV-1 replication comprising the use of a cyclin-dependent kinase inhibitor are provided.

### BACKGROUND

### 1. Field

The present disclosure relates to methods of inhibiting corneal neovascularization associated with herpes simplex virus-1 (HSV-1) infection of the eye.

### 2. Description of Related Art

The cornea is the highly transparent outer-most layer of the eye that provides a large portion of the eye's refractive power and shields against infection by pathogens. Since even the smallest of cells can affect visual acuity, normal corneal tissue lacks all vasculature and is immunologically privileged to reduce influx of inflammatory cells. Generally, in the absence of ocular disease and/or trauma, the lack of vasculature and inflammatory response of the cornea ensures visual acuity is maintained. However, under certain ocular situations, the lack of vascularization and/or suppressed inflammatory response may be compromised.

Angiogenesis is the formation of new blood vessels from pre-existing vessels during wound healing, pregnancy, tissue repair, and organ regeneration. Neovascularization is the formation of new blood vessels in previously avascular tissues. Both processes are mediated by the angiogenic factors vascular endothelial growth factor (VEGF) and basic fibroblast growth factor (bFGF). In the eye, corneal neovascularization and/or angiogenesis are pathogenic components of diabetic retinopathy, exudative macular degeneration, retinopathy of prematurity and HSV-1-induced corneal keratitis. This angiogenesis and/or neovascularization can lead to severe visual impairment from opacification of the cornea and from permanent changes to the neuronal architecture of the retina. Corneal neovascularization is a serious public health concern in the United States, affecting approximately 4% of the population.

Herpes simplex virus type 1 (HSV-1) infects approximately 90% of humans worldwide. HSV-1 infection and subsequent replication induce inflammation and neovascularization in the cornea of the eye leading to corneal blindness. In the United States, HSV infection is the leading cause of infection-induced blindness with 20,000 new cases reported each year. The most common treatment for ocular herpes infection is a combination of steroids to reduce neovascularization and antiviral drugs such as acyclovir or triflurothymidine (TFT) to reduce viral replication; however, treatment with corticosteroids increases the risk of glaucoma and cataracts. Thus, there exists a need to develop better interventions for this disease.

Recently, it was reported that subconjunctival administration of a monoclonal antibody raised against the angiogenic factor VEGF led to regression of corneal neovascularization in a rabbit model of herpetic stromal keratitis (HSK). In another study, HSV-1 was shown to induce lymphangiogenesis of the cornea through the stimulation of VEGF-A production. These studies suggest that by blocking pathways leading to VEGF production or by inhibiting signaling pathways downstream of VEGF, the negative effects of ocular neovascularization may be inhibited or reversed.

Cyclin-dependent kinases (CDKs) are typically involved in cell cycle control. Cyclin-dependent kinase 9 (CDK9) along with its partner, cyclin T1, comprise the positive-transcription elongation factor b (P-TEFb). CDK9 was first discovered as a host factor required for the transcription of full-length HIV RNA. CDK9 and cyclin T1 function by phosphorylating the C-terminal domain of RNA polymerase II (RNAPII) on serine 2 residues. When RNAPII is hyper-phosphorylated by CDK9, productive transcriptional elongation occurs. Much has been learned about the regulation of CDK9 using the pharmacological inhibitors flavopiridol (FP) and 5,6-Dichlorobenzimidazole-1-β-D-ribofuranoside (DRB). Both FP and DRB are characterized as compounds that inhibit the production of long but not short mRNA transcripts, and both FP and DRB are non-specific inhibitors of cellular kinases at high concentrations, but FP is specific for CDK9 at lower concentrations.

Ongoing clinical trials examining the use of FP in several types of cancer have shown little toxicity in vivo suggesting that it might be a suitable alternative for current ocular therapies. However, the role of CDKs in herpesvirus infection of the eye or on the efficacy of CDK9 inhibitors in preventing HSV-1 associated ocular neovascularization and its negative consequences has not been investigated.

The solution to this technical problem is provided by the embodiments characterized in the claims.

### BRIEF SUMMARY

The present application relates to methods of preventing and/or treating HSV-1-associated ocular neovascularization comprising administering a cyclin-dependent kinase inhibitor. In a more specific embodiment, methods of preventing and/or treating HSV-1-associated corneal neovascularization comprising administering a cyclin-dependent kinase inhibitor are provided.

The present application provides methods of inhibiting angiogenesis comprising the use of a cyclin-dependent kinase inhibitor. In a preferred embodiment, the cyclin-dependent kinase inhibitor is a CDK9 inhibitor. In a more preferred embodiment, the CDK9 inhibitor is flavopiridol.

The present application also provides methods of reducing endothelial cell migration comprising the use of a cyclin-dependent kinase inhibitor. In a preferred embodiment, the cyclin-dependent kinase inhibitor is a CDK9 inhibitor. In a more preferred embodiment, the CDK9 inhibitor is flavopiridol.

The present application also provides methods of reducing endothelial cell invasion comprising the use of a cyclin-dependent kinase inhibitor. In a preferred embodiment, the cyclin-dependent kinase inhibitor is a CDK9 inhibitor. In a more preferred embodiment, the CDK9 inhibitor is flavopiridol.

The present application also provides methods of reducing endothelial tubule formation comprising the use of a cyclin-dependent kinase inhibitor. In a preferred embodiment, the cyclin-dependent kinase inhibitor is a CDK9 inhibitor. In a more preferred embodiment, the CDK9 inhibitor is flavopiridol.

The present application additionally provides methods of reducing HSV replication comprising the use of a cyclin-dependent kinase inhibitor. In a preferred embodiment, the cyclin-dependent kinase inhibitor is a CDK9 inhibitor. In a more preferred embodiment, the CDK9 inhibitor is flavopiridol.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1A shows flavopiridol and DRB inhibition of corneal neovascularization in comparison with TFT. Corneal neovascularization was determined in a masked fashion by slit lamp analysis on days 2, 4, 7, 9, 11, 14, 18, and 25 post-infection (PI) with 5×10⁵ PFU/eye of HSV-1 17syn+ using the formula A=(C×0.4×L×n)/2 where C=clock hours of neovascularization; 30° arc is 1 (360° circle with a total of 12); L is the length of the longest neovessel, and A is the area of neovascularization. Mice were treated with PBS (control), 1% TFT, 0.01% flavopiridol, or 0.1% DRB in sterile saline.

FIG. 1B shows flavopiridol and DRB inhibition of corneal neovascularization in comparison with TFT. The combined clinical score was determined based on epiphora (excess tearing), inflammation and discharge. 0=manifestations, +1=slight manifestations, +2=moderate manifestations, and +3=severe manifestations.

FIG. 1C shows flavopiridol and DRB inhibition of corneal neovascularization in comparison with TFT. Histological score (0-1) was calculated based on leukocytic infiltration and peripheral neovascularization of hematoxylin & eosin (H&E) stained slides of mouse eyes on day 25 PI.

FIG. 2A shows flavopiridol (100 nM) inhibits endothelial cell migration in vitro. The percent of cell migration across a transwell insert toward complete medium containing fetal bovine serum (FBS) was calculated based on migration in the absence of flavopiridol. The top chamber (0.8-mm pore size) was seeded with approximately 3×10⁵ fluorescently labeled human umbilical vein endothelial cells (HUVEC)/well containing 0-200 nM flavopiridol, incubated for 16 hours at 37° C. and counted on an inverted microscope.

FIG. 2B shows flavopiridol (100 nM) inhibits endothelial cell invasion in vitro. Approximately 2.5×10⁴HUVEC were serum-starved for 16 hours and were seeded onto BD-Biocoat transwell plates with 25 ng/mL of bFGF and VEGF in the bottom chamber and with 0-200 nM flavopiridol in the top chamber. After 12-16 hours, tubules were counted at low magnification and percentage of inhibition was expressed using VEGF control wells as 100%.

FIG. 2C shows flavopiridol (100 nM) inhibits angiogenesis (tubule formation) in vitro. The number of circular tubular structures formed by HUVEC under conditions of serum starvation followed by treatment with 25 ng/mL bFGF and VEGF and with 0-200 nM FP were quantitatively analyzed. Manual counting at low power fields was used to quantitate tubular structures and percentage of inhibition was expressed using VEGF control wells as 100%.

FIG. 2D shows flavopiridol (100 nM) inhibits angiogenesis in vivo. Collagen plugs containing 25 ng/mL bFGF and VEGF with and without 100 nM flavopiridol were placed onto mesh grids of equal size on the chorioallantoic membrane (CAM) of Day 10 chick embryos and incubated at 99.5° F. ex ovo. After 3 days, CAMs were photographed, and angiogenesis was quantified by counting the number of grids positive for new blood vessel growth in a blinded manner. Results are expressed as number of grids positive for blood vessels and as percent positive grids.

FIG. 2E shows flavopiridol (100 nM) does not significantly induce death of endothelial cells in vitro when cells are incubated without ECGS or FBS. When cells are grown in complete medium containing ECGS and FBS, there is a statistically significant but small decrease in cell survival. Cell viability values (mean±SEM) are expressed as % of the value of the complete medium control.

FIG. 3A shows the effect of flavopiridol to reduce HSV replication in vitro. Numbers of plaque-forming units (PFU) were determined in a standard plaque assay procedure using CV-1 cells.

FIG. 3B shows the effect of DRB to reduce HSV replication in vitro. Numbers of plaque-forming units (PFU) were determined in a standard plaque assay procedure using CV-1 cells.

FIG. 3C shows the effect of flavopiridol and DRB to reduce HSV replication in vivo. SYBR Green semi-quantitative real-time PCR was performed on DNA samples eluted from swabs taken from mouse eyes on days 2, 4, 7, 9, 11, 14, 18 and 25 post infection with 5×10⁵ PFU/eye HSV-1 17syn+ using primers specific for HSV-1 DNA polymerase.

FIG. 4 shows the effect of flavopiridol to reduce corneal neovascularization due to chemical burn in mice. Following chemical treatment of mouse eyes with 0.15N NaOH, control and treated eyes were scored for neovascularization using the formula A=(C×0.4×L×pi)/2; where C=clock hours of neovascularization; 30° arc is 1 (360° L is the length of the longest neovessel, and A is the area of neovascularization. Animals were scored using a slit-lamp microscope, and images representative of each group were taken. One day post injury, flavopirodol reduced neovascularization to the level observed in undamaged and in damaged but prednisone-treated eyes.

FIG. 5A shows that flavopiridol does not block global transcription at concentrations that reduce angiogenesis in vitro. Vein endothelial cells (VEC) were activated with phorbol myristate acetate (PMA) and incubated with membrane-permeable 5-bromouridine (BrU) that incorporates into RNA transcribed during the incubation period. A commercially available antibody against 5-bromo-deoxy-uridine was then used to quantify BrU-containing RNA. Cells were analyzed by immunofluorescence staining (not shown) and by flow cytometry for the presence of incorporated BrU to quantify the level of global transcription. Treatment of cells with RNase A, but not with DNase, decreases BrU staining, indicating that BrU incorporated into RNA but not DNA. Additional controls include no antibody and no BrU.

FIG. 5B shows that flavopiridol at 100 nM differentially regulates gene expression rather than blocking transcription universally. Serum-starved, PMA-treated VEC were incubated in the presence of 50 nM-1 mM flavopiridol. BrU incorporation was only slightly reduced by 50 nM-300 nM flavopiridol signifying that low doses of flavopiridol do not decrease total cellular transcription. Higher concentrations (600 nM-1 mM) of flavopiridol significantly (p<0.05 compared with untreated cells) reduced BrU incorporation into RNA.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

As used herein, the terms “neovascularization” and “angiogenesis” are used interchangeably. Neovascularization and angiogenesis refer to the generation of new blood vessels into cells, tissue, or organs. The control of angiogenesis is typically altered in certain disease states and, in many cases, the pathological damage associated with the disease is related to altered, unregulated, or uncontrolled angiogenesis. Persistent, unregulated angiogenesis occurs in a multiplicity of disease states, including those characterized by the abnormal growth by endothelial cells, and supports the pathological damage seen in these conditions including leakage and permeability of blood vessels.

By “ocular neovascular disorder” is meant a disorder characterized by altered or unregulated angiogenesis in the eye of a patient. Exemplary ocular neovascular disorders include optic disc neovascularization, iris neovascularization, retinal neovascularization, choroidal neovascularization, corneal neovascularization, vitreal neovascularization, glaucoma, pannus, pterygium, macular edema, diabetic retinopathy, diabetic macular edema, vascular retinopathy, retinal degeneration, uveitis, inflammatory diseases of the retina, and proliferative vitreoretinopathy.

The term “treating” a neovascular disease in a subject or “treating” a subject having a neovascular disease refers to subjecting the subject to a pharmaceutical treatment, e.g., the administration of a drug, such that at least one symptom of the neovascular disease is decreased. Accordingly, the term “treating” as used herein is intended to encompass curing as well as ameliorating at least one symptom of the neovascular condition or disease. Accordingly, “treating” as used herein, includes administering or prescribing a pharmaceutical composition for the treatment or prevention of an ocular neovascular disorder.

By “patient” is meant any animal. The term “animal” includes mammals, including, but is not limited to, humans and other primates. The term also includes domesticated animals, such as cows, hogs, sheep, horses, dogs, and cats.

By “cyclin-dependent kinase inhibitor” or “CDK inhibitor” is meant any compound that reduces, or inhibits, either partially or in full, the activity or production of a CDK. A CDK inhibitor may directly or indirectly reduce or inhibit a specific CDK, such as CDK9.

By “an amount sufficient to suppress a neovascular disorder” is meant the effective amount of a compound required to treat or prevent a neovascular disorder or symptom thereof. The “effective amount” of compound used to practice the present invention for therapeutic treatment of conditions caused by or contributing to the neovascular disorder varies depending upon the manner of administration, anatomical location of the neovascular disorder, the age, body weight, and general health of the patient. Ultimately, a physician or veterinarian will decide the appropriate amount and dosage regimen. Such amount is referred to as an amount sufficient to suppress a neovascular disorder.

Ocular Neovascularization

The subject disclosure features, in one aspect, new methods and treatments for ocular neovascularization using an inhibitor of cyclin-dependent kinase. In some embodiments, ocular neovascular disorders amenable to treatment or suppression by cyclin-dependent kinase inhibitors include ischemic retinopathy, iris neovascularization, intraocular neovascularization, age-related macular degeneration, corneal neovascularization, retinal neovascularization, choroidal neovascularization, diabetic retinal ischemia, or proliferative diabetic retinopathy. In a preferred embodiment, the ocular neovascularization is corneal neovascularization. Corneal neovascularization can be induced by several factors, including, but not limiting to, irritation of the eye (i.e. contact lens-induced corneal neovascularization) and viral infection (i.e. herpesvirus infection).

Neovascularization (NV) of the cornea represents a state of disease secondary to a variety of corneal insults, including contact lens wear. Diseases associated with corneal neovascularization that can be treated include, but are not limited to, corneal graft rejection, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, herpes simplex infections, herpes zoster infections, protozoan infections, trauma, rheumatoid arthritis, systemic lupus, polyarteritis, Wegener's sarcoidosis, scleritis, Stevens-Johnson disease, pemphigoid, radial keratotomy, and corneal graft rejection.

Conjunctival neovascularization that can be treated include, but are not limited to, pinguecula, pterygium, squamous cell carcinoma, pre-malignant lesions, and scarring.

Skin and Eyelids

Skin and eyelid lesions associated with angiogenesis that can be treated include, but are not limited to, squamous cell carcinoma, basal cell carcinoma, angioma, haemangioma, scar, granuloma, and other tumors of skin, eyelids, and orbit.

Retinal/Choroidal Neovascularization

With choroidal neovasculatization, abnormal blood vessels stemming from the choroid grow up through the retinal layers. Diseases associated with retinal/choroidal neovascularization that can be treated include, but are not limited to, diabetic retinopathy, macular degeneration, retinopathy of prematurity, sickle cell retinopathy, myopic degeneration, histoplasmosis, sarcoidosis, angioid streaks, syphilis, pseudoxanthoma elasticum, Paget's disease, vein occlusion, artery occlusion, carotid obstructive disease, chronic uveitis/vitritis, mycobacterial infections, Lyme disease, systemic lupus erythematosus, Eales' disease, choroidal angioma, retinal angioma, ocular melanoma, Behcet's disease, retinitis or choroiditis, presumed ocular histoplasmosis, Best's disease, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the iris), neovascularization of angle, neovascular glaucoma and diseases caused by the abnormal proliferation of fibrovascular or fibrous tissue including all forms of proliferative vitreoretinopathy, whether or not associated with diabetes.

Viral-Induced Ocular Neovascularization

An advantage of the methods described herein include a treatment for viral-induced ocular neovascularization that is effective at both reducing viral replication and neovascularization simultaneously, thus reducing cost to the patient and reducing the number of medications required to treat the patient. Also, by targeting cellular kinases rather than viral targets, viral escape mechanisms may be avoided.

In a specific embodiment, the viral-induced ocular neovascularization is viral-induced corneal neovascularization.

In a more specific embodiment, the neovascularization is induced by herpes simplex virus (HSV) infection. In a preferred specific embodiment, the HSV infection is HSV1 infection.

Cyclin-Dependent Kinase Inhibitors

There are several cyclin-dependent kinase inhibitors known in the art, ranging from pan-CDK inhibitors to highly selective inhibitors of specific CDKs. In one embodiment of the invention, the use of any CDK inhibitor is contemplated. In a preferred embodiment, the CDK inhibitor is a pan-CDK inhibitor that inhibits CDK9. In an additional preferred embodiment, the CDK inhibitor is a highly selective inhibitor of CDK9. In a most preferred embodiment, the CDK inhibitor is flavopiridol or dichlorobenzimidazole-1-β-D-ribofuranoside (DRB).

Modes of Administration

The cyclin-dependent kinase inhibitor can be administered via any pharmaceutically acceptable method, including but not limited to, intravenously, subcutaneously, intramuscularly, topically, orally, parenterally, or by inhalation.

Pharmaceutical compositions suitable for parenteral administration may comprise one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed include water, saline, balanced salt solution, ethanol, polyols (such as glycerol, propylene glycol, polyethylene glycol,), and suitable mixtures thereof. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol, and sorbic acid. It may also be desirable to include isotonic agents, such as sugars or sodium chloride into the compositions. In addition, prolonged absorption of the injectable pharmaceutical form may be brought about by the inclusion of agents which delay absorption such as polyethylene glycol (PEG), aluminum monostearate and gelatin.

The following Examples describe exemplary embodiments of the invention. These Examples should not be interpreted to encompass the entire breadth of the invention.

### EXAMPLES

Flavopiridol reduces HSV-1-associated ocular neovascularization and pathogenesis as effectively as triflurothymidine in a mouse model.

Methods

Materials

bFGF and VEGF were obtained from R&D Systems (Minneapolis, Minn.). Flavopiridol was purchased from Sigma Chemical Company (St. Louis, Mo.). Triflurothymidine (TFT) was purchased from the Louisiana State University School of Veterinary Medicine. Celltracker green and Calcein AM were purchased from Life Technologies (Carlsbad, Calif.).

Animal Models and Analysis

All experimental procedures were performed in accordance with the ARVO Resolution for the Use of Animals in Ophthalmic and Vision Research and were approved by the Xavier University Animal Care and Use Committee. Female 20 g Balb/c mice were obtained from Charles River Laboratories. Mice were allowed to equilibrate to their surroundings for 2 weeks and then were anesthetized by intramuscular administration of xylazine (6.6 mg/kg of body weight) and ketamine (100 mg/kg of body weight). The corneas were mildly scarified in a 2×2 cross-hatch pattern and each cornea was inoculated with 5×10⁵ PFU of strain HSV 17syn+. Drops (PBS control, 1% triflurothymidine (TFT), 0.01% flavopiridol and 0.1% dichlorobenzimidazole-1-β-D-ribofuranoside (DRB)) were applied 4 times daily for 14 days to 10 mice in each group. DNA was collected by swabbing the eyes with filter paper, and the eyes were examined with a slit-lamp on PI days 2, 4, 7, 9, 11, 14, 18 and 25. Average neovascularization/eye was scored using the formula A=(C×0.4×L×π)/2 where C=clock hours of neovascularization; 30° arc is 1 (360° circle with a total of 12); L is the length of the longest neovessel, and A is the area of neovascularization. Students scored (by eye) the animals on the days indicated in a masked fashion using the slit-lamp, and images representative of each group were taken. Clinical scores based on epiphora, inflammation and discharge were calculated where 0=no manifestations, +1=slight manifestations, +2=moderate manifestations, and +3=severe manifestations. Mice were sacrificed with CO₂ on day 25 post-infection, and eyes were harvested, sectioned, stained with hematoxylin and eosin (H&E) for histological analysis based on peripheral neovascularization and leukocytic infiltration where 0=clear eyes, 0.5=either peripheral neovascularization or leukocytic infiltration, and 1=both peripheral neovascularization and leukocytic infiltration. Ex ovo chick embryo chorioallantoic membrane collagen angiogenesis assays were performed using methods as previously described (Deryugina E I and Quigley J P. Chapter 2. Chick embryo chorioallantoic membrane models to quantify angiogenesis induced by inflammatory and tumor cells or purified effector molecules. Methods Enzymol. 444:21-41. 2008). Briefly, fertilized embryos (Charles River Laboratories, Charleston, S.C.) were incubated at 37.5° C. for 3 days, removed from their shell using a Dremel tool and placed into a covered weighing boat for 7 further days of incubation. Solidified 30 μL onplants containing 2.1 mg/mL rat tail collagen (BD Biosciences, Bedford, Mass.) and 10 ng bFGF and 30 ng VEGF in the presence or absence of flavopiridol were placed on the CAM over two pieces of nylon mesh approximately 0.5 cm². Four collagen onplants were added per egg on at least 3 separate eggs. After three additional days of incubation, images were taken of each plug using a mini-Vid camera (LW Scientific; Lawrenceville, Ga.) and quantified in a masked fashion based on the percentage of grids that were positive for newly formed blood vessels.

Virus and Cell Culture

The HSV-1 strain employed was 17 syn+. Before inoculation, numbers of plaque-forming units (PFU) were determined in a standard plaque assay procedure using CV-1 cells. Pooled primary human umbilical vein endothelial cells (HUVEC) were obtained from Lonza (Basel, Switzerland) and were cultured in Medium 199 (Life Technologies, Carlsbad, Calif.) supplemented with 20% heat-inactivated FBS, penicillin G (100 U/ml), streptomycin (100 mg/ml), 2 mM L-glutamine and 1:100 endothelial cell growth supplement (ECGS, BD Biosciences, Bedford, Mass.) on tissue culture plates coated with 0.2% gelatin up to passage 6. Cells were sub-passaged or harvested when they reached ˜80% confluence. For periods of serum-deprivation, HUVEC were incubated for 4 or 16 hours in serum-starvation medium (SSM) containing Medium 199 without phenol red or ECGS but containing 0.5% FBS unless otherwise indicated. In vitro viral inhibition assays with CV-1 cells were performed as previously described (Gong E Y. Methods for screening and profiling inhibitors of herpes simplex viruses. Methods Mol Biol. 1030:303-313. 2013).

Kinase Assays

RNAPII CTD kinase assays were performed as previously described (Herrmann C H et al. Tat-associated kinase, TAK, activity is regulated by distinct mechanisms in peripheral blood lymphocytes and promonocytic cell lines. Journal of Virology. 72:9881-9888. 1998). Following treatment, HUVEC were isolated by scraping in EBC buffer (50 mM Tris pH 8.0, 120 mM NaCl, 0.5% NP-40, 0.03% SDS, 5 mM DTT and Complete Mini Protease Inhibitor Cocktail (Roche Applied Science, Indianapolis, Ind.)) or in RIPA buffer (50 mM Tris pH 8.0, 150 mM NaCl, 1% NP-40, 0.1% SDS) containing complete Mini Protease Inhibitor Cocktail (Roche). Following a 30 minute incubation at 4° C., DNA was sheared using a 27 gauge needle. 1 μL of DNase+RNase mix (10 mg/mL RNase A+20 U/μL DNase I) was added and protein concentrations were measured by the Bradford method (Sigma). CDK9 was then affinity precipitated from whole cell extracts standardized for protein concentration using recombinant HIV Tat-2 expressed as a GST fusion protein with glutathione-sepharose beads. Kinase reactions were carried out at room temperature for 30 minutes in reaction buffer (25 μL TKB/Mg10 (50 mM Tris pH 7.4, 10 mM MgCl₂, 5 mM DTI), 2.5 mM MnCl₂ 200 ng GST-CTD, 5 μM ATP and 5-10 μCi γ ³²P-ATP). Beads were boiled in 2× Laemmli sample buffer for 8 minutes, electrophoresed on 9% polyacrylamide gels and visualized on a phosphorimager or by autoradiography.

EC Migration/Invasion Assay

Migration/invasion assays were performed using 24-well, 8-μm pore size BD Biocoat Matrigel migration chambers according to the manufacturer's instructions (BD Biosciences, San Jose, Calif.). The bottom chambers were filled with 800 μL of medium supplemented with complete medium containing FBS (migration) or 25 ng/mL VEGF and bFGF (invasion) in the presence or absence of flavopiridol. The top chamber was seeded with approximately 2.5×10⁴ HUVEC cells/well in 200 μL containing 0-200 nM flavopiridol. Cells were allowed to migrate for 16 hours at 37° C. Following incubation, cotton swabs were used to remove the cells on the top surface of the membrane, and cells on the bottom side of the membrane were fixed with 4% formaldehyde for 30 min, washed 3-5 times with PBS, and then stained with 10 μM Calcein AM (migration) or 10 μM Celltracker green-labeled (invasion). Migrating or invading cells from three repeats in two independent experiments were then counted using an inverted microscope. Data are expressed as mean percent migration and mean percent invading cells compared with untreated cells±SEM.

In Vitro Tubule Formation Assay

Growth factor-reduced Geltrex (Life Technologies, Grand Island, N.Y.) was thawed overnight on ice. Each well of 96-well plated was coated at 40° C. with 50 μL Geltrex and incubated at 37° C. for 30 min. HUVEC were serum-starved for 16 hours, harvested and approximately 1×10⁴ cells/well were seeded with 25 ng/mL of bFGF and VEGF and with 0-200 nM flavopiridol. After 12-16 hours, tubule formation was assessed with an inverted photomicroscope and the images were photographed using Olympus U-RLF-T microscope. Manual counting at low power fields was used to quantitate tubular structures and percentage of inhibition was expressed using VEGF control wells as 100%. Results represent one triplicate repeat of three independent experiments with similar results.

Cell Death Assays

HUVEC were grown in complete medium, plated at 5000 cells/well in a 96 well plate and then treated for 24 hours with media as indicated. Complete medium contained 10% FBS and 1× Endothelial Growth Supplement (ECGS). Serum starvation media contained no FBS or ECGS. These media were tested in the presence and absence of 100 nM FP. After treatment, the media was replaced with 100 μL of complete medium with 10 μL of 12 mM tetrazolium salt MTT for 4 hours at 37° C. 100 μL of sodium dodecyl sulfate (SDS)-HCl solution was then added, and the cells were incubated for 4 additional hours at 37° C. The amount of violet crystals reflecting cellular growth and viability was determined by absorbance at 570 nm. Each sample was assayed at least three times.

Determination of Copy Number by Real-Time PCR

Real-time PCR reactions were performed in a 20 μL volume containing a solution of 1×SYBR green supermix (Bio-Rad), 1 μL of forward primer, 1 μL of reverse primer, and 2 μL of DNA eluted from frozen ocular swabs with a PureLink Viral RNA/DNA Mini Kit (Life Technologies) according to the manufacturer's instructions. A four-step protocol was used: for amplification of viral DNA denaturation, 3 minutes at 95° C.; amplification and quantification, 45 cycles for 10 seconds at 95° C., for 30 seconds at 55° C. and 72° C. for 10 seconds; melting curve, 60 to 95° C. with a heating rate of 0.5° C. per second; followed by cooling using a iQ5 thermocycler (Bio-Rad). A single-peak melting curve was observed for each gene product. The HSV DNA Pol Forward Primer was 5′-AGA GGG ACA TCC AGG ACT TTG T-3′ (SEQ ID NO: 1), and the HSV DNA Pol Reverse Primer was 5′-CAG GCG CTT GTI′ GGGT GTA C-3′ (SEQ ID NO: 2) (IDT, Inc, Coralville, Iowa). CT values were normalized with HSV DNA polymerase control in a PCR2.1 plasmid to determine viral copy number.

Statistics

Data are presented as the means±standard errors of the mean. Statistical significances were determined by one-way analysis of variance (ANOVA) followed by Dunnet's Multiple Comparison test (GraphPad Prism, San Diego, Calif.) where *=p<0.05, **=p<0.01, ***=p<0.001, and ****=p<0.0001.

### Example 1: Flavopiridol and DRB Lower Corneal Neovascularization and Other Clinical Symptoms of Ocular HSV-1 Infection to Levels Observed in TFT-Treated Mice

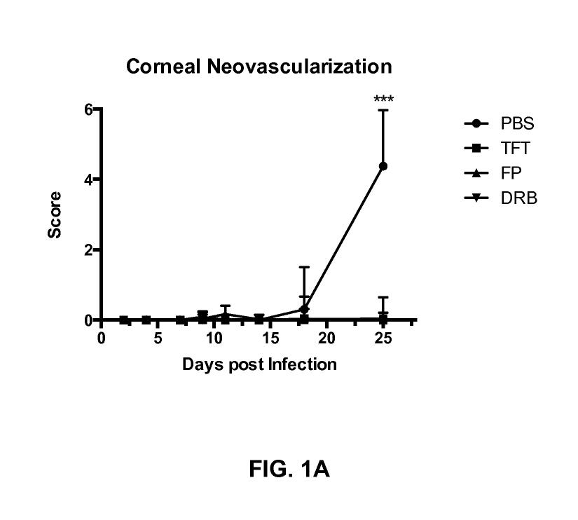

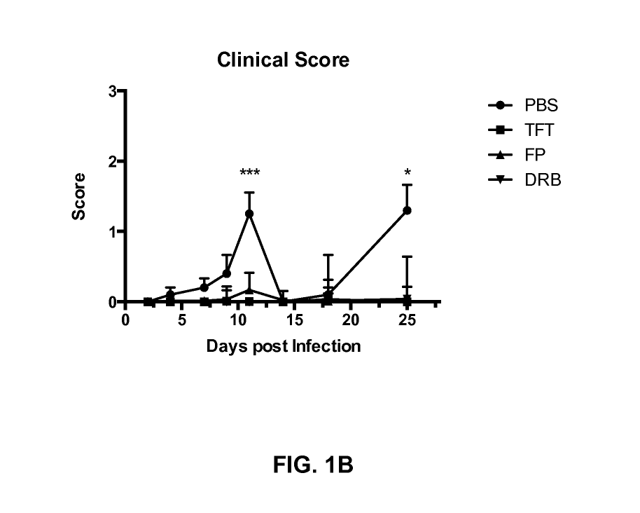

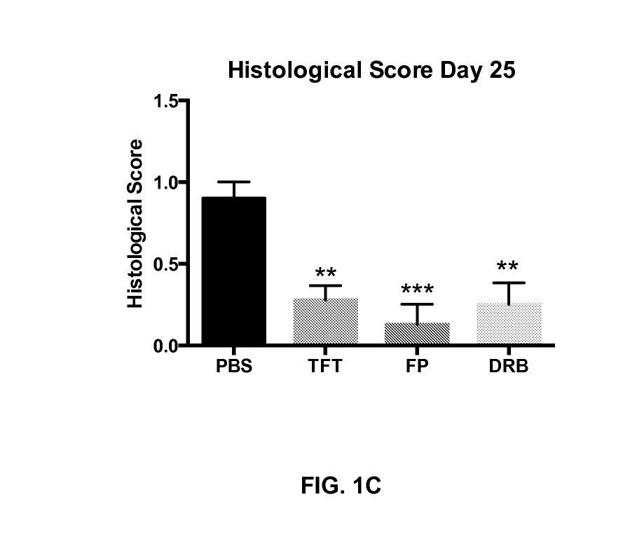

The flavopiridol and dichlorobenzimidazole-1-β-D-ribofuranoside (DRB) formulation that we employed was safe and well-tolerated in mouse and rabbit eyes. This was assessed with the dose and frequency that was used in the chemotherapeutic study of HSV-1 corneal neovascularization. FIG. 1A presents the results of slit-lamp examination (SLE) scores of the corneas of the four treatment groups beginning on post-inoculation day 2 and continuing to 25 days following infection. The triflurothymidine (TFT; 0.0±0.0; n=10), flavopiridol (0.039±0.176, n=10) and DRB (0.042±0.60, n=10) scores were significantly lower than those from the PBS control group (4.37±1.59, n=5) on day 25.

FIG. 1B is the combined clinical score for the treatment groups based on epiphora (excess tearing), inflammation, and discharge. Significant differences were noted between PBS control and flavopiridol, DRB, and TFT-treated mice on days 11 and 25. On day 11, the TFT (0.002±0.41), flavopiridol (0.169±0.248), and DRB (0.00±0.0) scores were significantly lower than those from the PBS control group (1.25±0.3). On day 25, the TFT (0.00±0.00), flavopiridol (0.039±0.177), and DRB (0.042±0.60) scores were also significantly lower than those from the PBS control group (1.3±0.37).

To confirm the results in FIGS. 1A and 1B, sections of one cornea from each mouse was sectioned, stained with H&E and analyzed for leukocytic infiltrate and peripheral neovascularization of the cornea. On a scale from 0-1, the TFT (0.278±0.088), flavopiridol (0.125±0.125), and DRB (0.25±0.134) scores were significantly lower than those from the PBS control group (0.90±0.10) (FIG. 1C). 100% of PBS-treated mouse eyes exhibited leukocytic infiltration compared with 30% for TFT, 0% for flavopiridol, and 10% for DRB-treated eyes. 60% of PBS-treated mouse eyes exhibited corneal neovascularization compared with 0% for TFT, flavopiridol, and DRB-treated eyes.

### Example 2: Flavopiridol Significantly Reduces Endothelial Cell Migration In Vitro

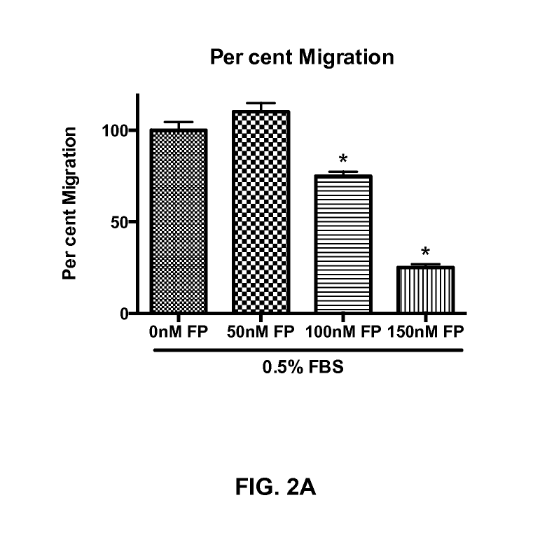

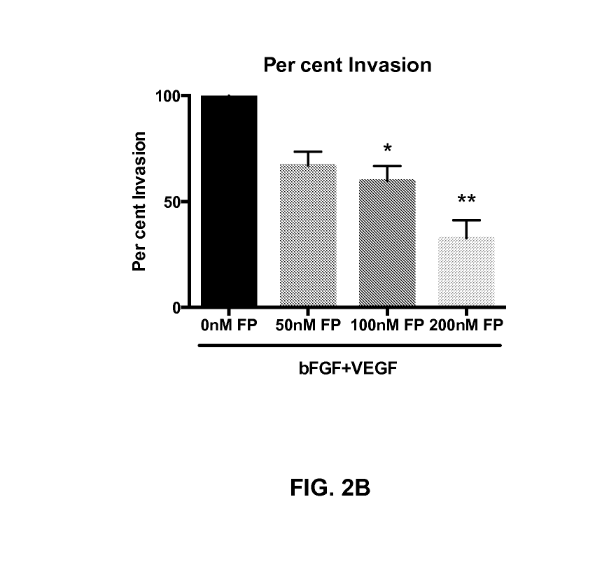

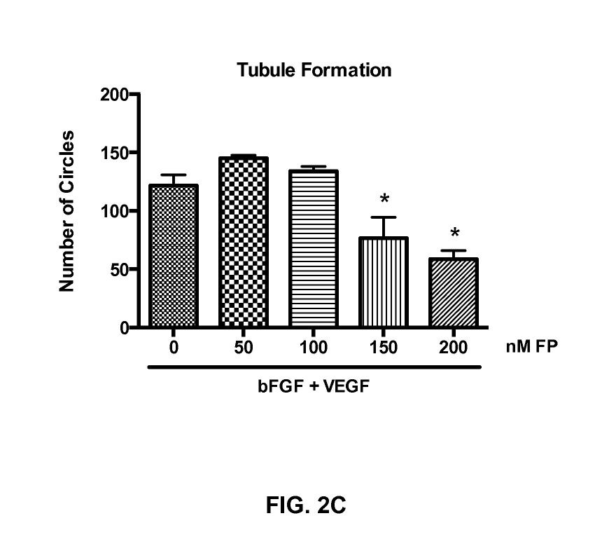

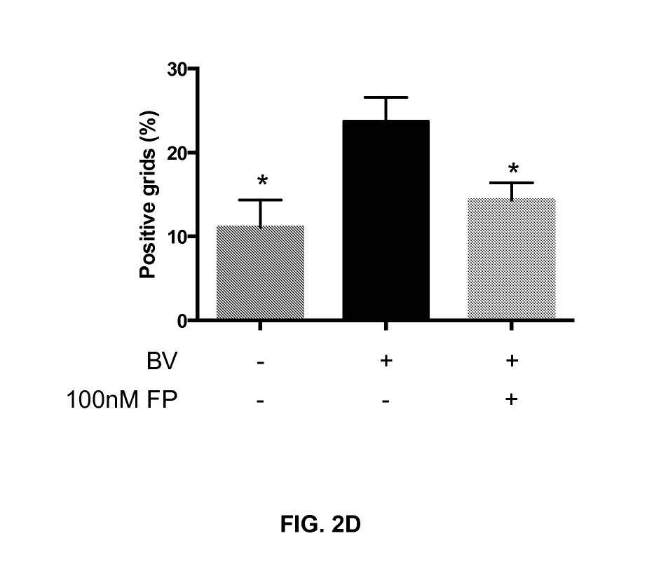

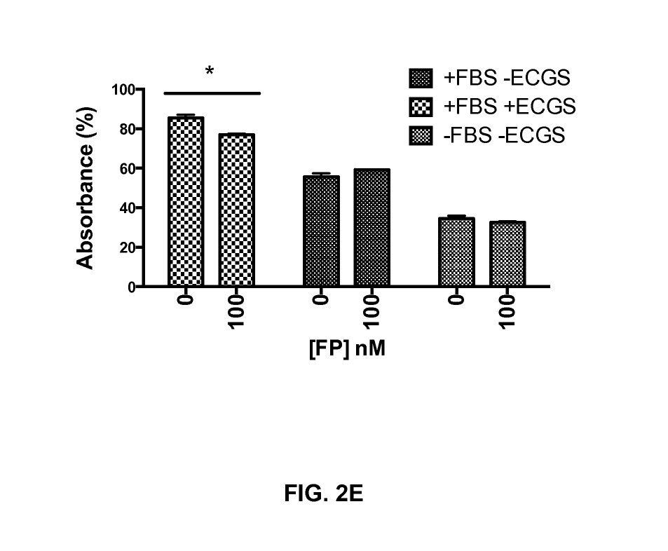

Cell migration induced by chemoattractant angiogenic factors such as bFGF and VEGF is an important step in the angiogenic process. We therefore examined the ability of flavopiridol to inhibit HUVEC migration induced by 0.5% FBS-containing media, since migration is relevant to the ability of endothelial cells to form new blood vessels. We analyzed HUVEC migration using a standardized transwell assay to assess the ability of flavopiridol to inhibit bFGF- and VEGF-induced migration, since such migration is relevant to pathological neovascularization induced by ocular HSV-1 infection. We found that the inhibitory effect of flavopiridol is dose dependent with significant inhibition occurring at 100 nM (74.99%±2.35) and 150 nM flavopiridol (25.08±1.79) compared with control (100%±4.5) (FIG. 2A). Data presented as percent fluorescence normalized to control untreated cells (100% migration).

### Example 3: Flavopiridol Significantly Reduces Endothelial Cell Invasion In Vitro

In addition to migration, new blood vessel formation requires that endothelial cells invade through extracellular matrix (ECM) surrounding the existing vasculature. Inhibition of invasion therefore inhibits angiogenesis. To evaluate the ability of flavopiridol to reduce VEGF- and bFGF-induced HUVEC invasion, we utilized a Matrigel transwell assay. Celltracker green-labeled HUVEC that successfully degraded the Matrigel and crossed into the lower chamber containing 25 ng/mL VEGF and bFGF in the presence or absence of FP were counted and expressed as a percent of untreated control cells (FIG. 2B). Compared to HUVEC treated without flavopiridol, flavopiridol significantly inhibited VEGF- and bFGF-induced cell migration and invasion at a dose range of 50-150 nM. 100%±11.95 invading cells was reduced to 67.08%±6.50 with 50 nM flavopiridol, 59.74%±6.98 with 100 nM flavopiridol and 32.64%±8.58 with 200 nM flavopiridol.

### Example 4: Flavopiridol Significantly Reduces Endothelial Cell Tubule Formation In Vitro

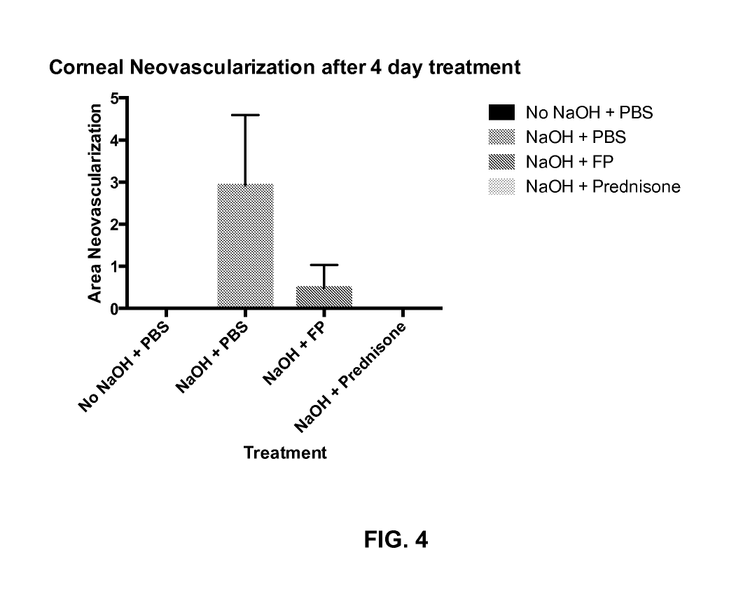

Following migration from the vessel of origin and invasion through the ECM, ECs form capillary tubules. We assessed whether flavopiridol could impair EC differentiation into tubule like structures using a standardized tubule formation assay (FIG. 4A). The number of circular nodes surrounded by tubular structures was quantitatively analyzed in a masked fashion. Flavopiridol significantly inhibited the ability of HUVEC to form capillary tubules compared to VEGF and bFGF control in a concentration-dependent manner in the range of 150-200 nM (FIG. 2C). 100.3±3.285 (0 nM flavopiridol) was significantly reduced to 55.33±9.70 by 150 nM flavopiridol. 50 nM flavopiridol (114.7±7.17) and 100 nM flavopiridol (97.67±5.36) did not produce statistically different results compared to control treated cells.

### Example 5: Flavopiridol Significantly Reduces In Vivo Angiogenesis in a Chick Chorioallantoic Membrane (CAM) Model

An ex ovo chick CAM model was used to determine whether 100 nM flavopiridol significantly inhibited angiogenesis in vivo. Ex ovo chick embryo chorioallantoic membrane collagen angiogenesis assays were performed. Fertilized embryos (Charles River Laboratories, Charleston, S.C.) were incubated at 37.5° C. for 3 days, removed from their shell using a Dremel tool and placed into a covered weighing boat for 7 further days of incubation. Solidified 30 μL onplants containing 2.1 mg/mL rat tail collagen (BD Biosciences, Bedford, Mass.) and 10 ng bFGF and 30 ng VEGF in the presence or absence of FP were placed on the CAM over two pieces of nylon mesh approximately 0.5 cm². Four collagen onplants were added per egg on at least 3 separate eggs. After three additional days of incubation, images were taken of each plug using a mini-Vid camera (LW Scientific; Lawrenceville, Ga.) and quantified in a masked fashion based on the percentage of grids that were positive for newly formed blood vessels. bFGF/VEGF-containing collagen plugs significantly increased angiogenesis (23.66±2.91) compared with vehicle (11.06±3.32), and this was reduced by the inclusion of 100 nM flavopiridol in the bFGF/VEGF-containing plugs (14.33±2.10) (FIG. 2D).

### Example 6: Flavopiridol does not Significantly Reduce Endothelial Cell Viability at Lower Concentrations that Inhibit Angiogenesis

To determine whether flavopiridol induced significant cell death in endothelial cells as has been reported in several cancer cell lines, we performed an 3-(4,5-dimethylthiazol-2-yl)-2,5-diphenyltetrazolium bromide (MIT) cellular metabolic activity assay using 100-800 nM doses of flavopiridol under conditions of serum starvation used to mimic the angiogenic process in vitro. In this assay, absorbance did not significantly change from 55.7%±1.87 in control cells to 59.3±0.058 in cells treated with 100 nM flavopiridol when the cells were incubated without 1× Endothelial Growth Supplement (ECGS) but with FBS. Statistical significance was reached at 200 nM flavopiridol with absorbance readings of 45.17±0.48. Absorbance decreased from 34.53±1.51 in control cells to 32.5±0.61 in cells treated with 100 nM flavopiridol when the cells were incubated without FBS or ECGS and became significant at 200 nM (27.63±0.50). Absorbance decreased 49.72% in media containing no FBS and no ECGS with the addition of 800 nM flavopiridol, and it decreased 58.76% in cells incubated in 800 nM flavopiridol with FBS but without ECGS (FIG. 2E).

### Example 7: Flavopiridol and DRB Reduce HSV Replication In Vitro

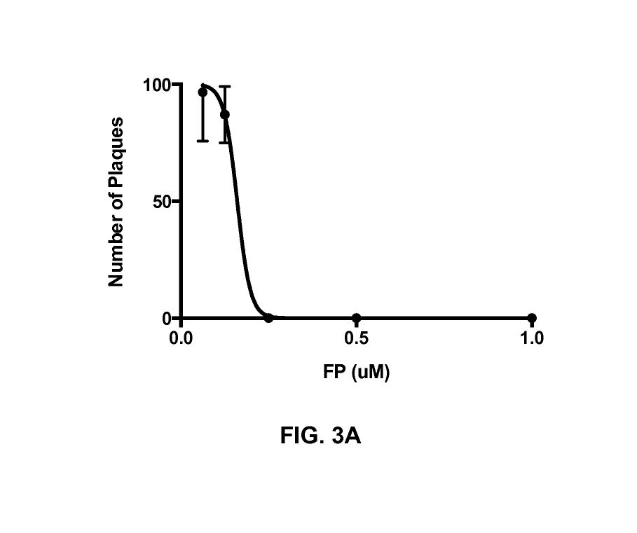

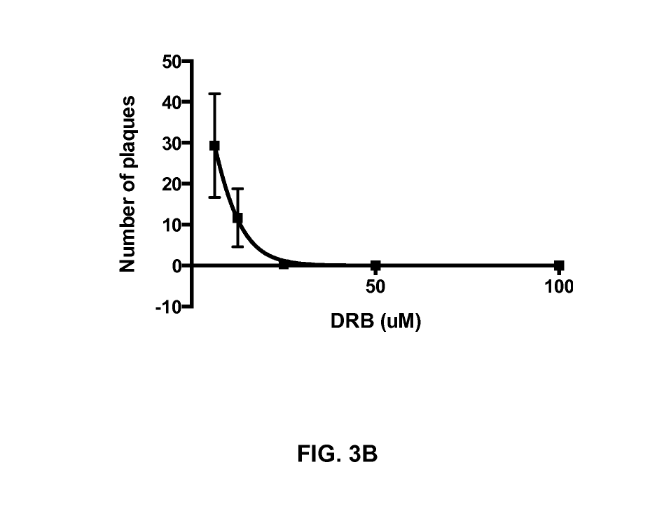

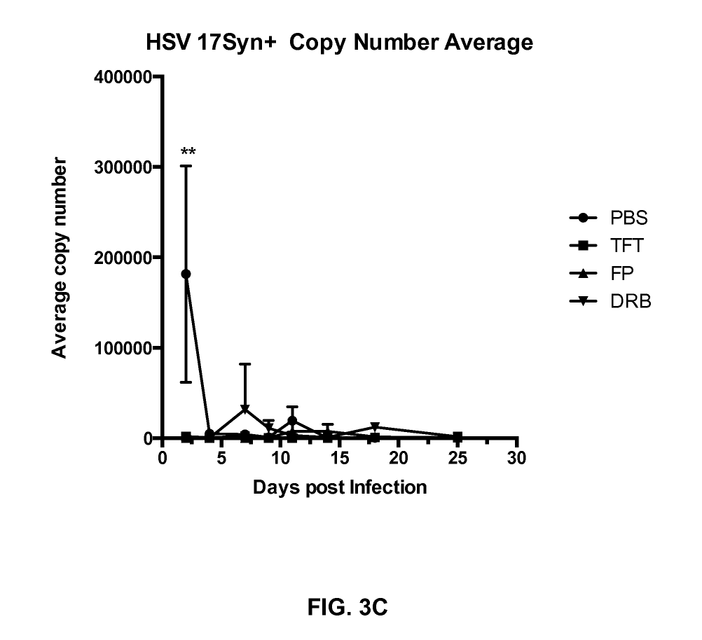

FIGS. 3A and 3B present the results of an in vitro viral inhibition assay using CV-1 cells to test whether flavopiridol and DRB inhibit viral replication. Flavopiridol significantly inhibited HSV-1 replication in an in vitro viral inhibition assay using CV-1 cells at 250 nM with an average IC₅₀ of 99.95 nM. DRB significantly reduced HSV-1 replication in CV-1 cells at a concentration of 25 μM with an average IC₅₀ of 86.7 μM.

### Example 8: Flavopiridol and DRB Reduce HSV Replication In Vivo

FIG. 3C presents the results of quantitative real-time PCR on swabs obtained from the four treatment groups beginning on post-inoculation day 2 and continuing to 25 days following infection. The TFT (0.0±0.0, n=10), flavopiridol (0.039±0.176, n=10), and DRB (0.042±0.60, n=10) scores were significantly lower than those from the PBS control group (4.37±1.59, n=5) on day 25.

### Example 9: Flavopiridol Reduces Neovascularization Due to Chemical Burn

To determine whether FP would reduce corneal neovascularization due to chemical injury in a mouse model system, female Balb C mice (7 per group) were treated in their right eyes with 0.15N NaOH for 15 seconds and the left eye was not treated with NaOH. Each group was then treated 5× daily for 4 additional days with either 0.1% BSA in phosphate-buffered saline (PBS) control, 0.01% FP or 1% prednisolone acetate (prednisone) ophthalmic solution in both eyes. Neovascularization was scored using the formula A=(C×0.4×L×pi)/2; where C=clock hours of neovascularization; 30° arc is 1 (360° L is the length of the longest neovessel, and A is the area of neovascularization. Animals were scored in a masked fashion using a slit-lamp microscope, and images representative of each group were taken. The longest possible length of a neovessel is 1.6 mm and the greatest possible arc is 12/12 hours.

Four days post injury, FP reduced neovascularization due to alkali burn to the level observed in undamaged and in damaged but prednisone-treated eyes (FIG. 4).

### Example 10: Flavopiridol does not Block Global Transcription at Concentrations that Reduce Angiogenesis In Vitro

Previous reports have indicated that low-dose FP does not block global transcription [Lu, et al. Mol Cancer Ther (2004) 3(7):861-872]. To determine whether FP blocked transcription globally at concentrations at which it reduces angiogenesis in vitro, HUVEC were activated with phorbol myristate acetate (PMA) and incubated with membrane-permeable 5-bromouridine (BrU) that incorporates into RNA transcribed during the incubation period. A commercially available antibody against 5-bromo-deoxy-uridine was then used to quantify BrU-containing RNA. Cells were analyzed by immunofluorescence staining (not shown) and by flow cytometry for the presence of incorporated BrU to quantify the level of global transcription.

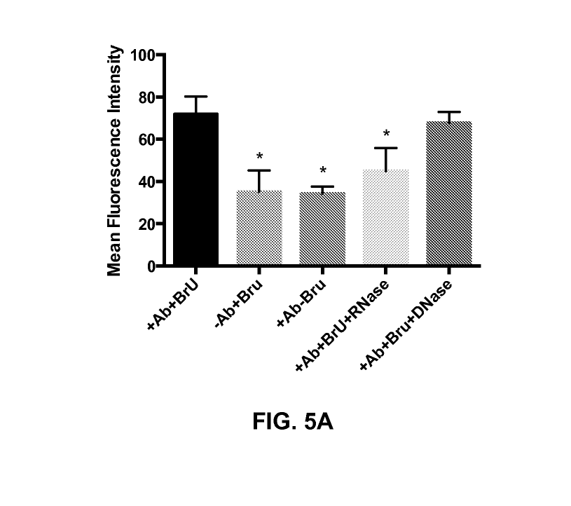

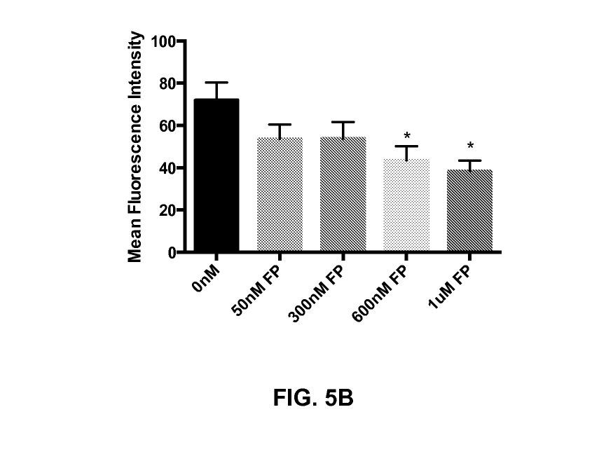

Treatment of cells with RNase A but not with DNase decreases BRU staining, indicating that BrU incorporated into RNA but not DNA. Additional controls include no antibody (-Ab) and no BrU (-BrU) (FIG. 5A). BrU incorporated linearly with respect to time into RNA over a 3 hour time course (Not shown). To confirm unpublished results from our laboratory indicating that 100 nM FP differentially regulates gene expression rather than blocking transcription universally, we incubated serum-starved, PMA-treated HUVEC in the presence of 50 nM-1 μM FP. BrU incorporation was only slightly reduced by 50 nM-300 nM FP signifying that low doses of FP do not decrease total cellular transcription. Higher concentrations (600 nM-1 μM) of FP significantly (p<0.05 compared with untreated cells) reduced BrU incorporation into RNA (FIG. 5B).

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method of preventing and/or treating HSV-1-associated ocular neovascularization in a patient in need thereof, the method comprising administering an effective amount of a cyclin-dependent kinase (CDK) inhibitor to said patient.

2. The method of claim 1, wherein the HSV-1-associated ocular neovascularization is HSV-1-associated corneal neovascularization.

3. The method of claim 1, wherein the CDK inhibitor is a CDK9 inhibitor.

4. The method of claim 3, wherein the CDK9 inhibitor is selected from the group consisting of flavopiridol and dichlorobenzimidazole-1-β-D-ribofuranoside (DRB).

5. A method of inhibiting angiogenesis in a patient in need thereof, the method comprising administering an effective amount of a cyclin-dependent kinase inhibitor to said patient.

6. The method of claim 5, wherein the CDK inhibitor is a CDK9 inhibitor.

7. The method of claim 6, wherein the CDK9 inhibitor is selected from the group consisting of flavopiridol and dichlorobenzimidazole-1-β-D-ribofuranoside (DRB).

8. A method of reducing HSV replication in a patient in need thereof, the method comprising administering an effective amount of a cyclin-dependent kinase inhibitor to said patient.

9. The method of claim 8, wherein the CDK inhibitor is a CDK9 inhibitor.

10. The method of claim 9, wherein the CDK9 inhibitor is selected from the group consisting of flavopiridol and dichlorobenzimidazole-1-β-D-ribofuranoside (DRB).

11-20. (canceled)
